(19) United States
(12) Patent Application Publication
(10) Pub. No.: US 2004/0097422 A1
(43) Pub. Date: May 20, 2004

(54) Methods of use for tripeptidyl peptidase II inhibitors as anticancer agents

(76) Inventors: Karl Munger, Newton, MA (US); Stefan M. Duensing, Boston, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/460,936

(22) Filed: Jun. 13, 2003

Related U.S. Application Data
(60) Provisional application No. 60/388,722, filed on Jun. 14, 2002.

(51) Int. Cl. A61K 38/06

(52) U.S. Cl. 514/18

(57) ABSTRACT

Methods are provided for abrogating tripeptidyl peptidase II (TPPII) activity and suppressing c-MYC induced abnormal centriole duplication. Methods of inhibiting TPPII using selective inhibitors such as butabindide provide a preventive or therapeutic strategy to target genomic instability, tumorigenic progression and chemotherapy resistance in tumors with overexpression of c-Myc.

### RELATED APPLICATION

This application claims the benefit of U.S. Provisional Application No. 60/388,722, filed Jun. 14, 2002, and which is hereby incorporated in its entirety herein.

### GOVERNMENT FUNDING

This invention was made in part with government support under grant CA66980 awarded by the National Institutes of Health. The government has certain rights in the invention.

### TECHNICAL FIELD

The invention herein relates to methods and compositions that are inhibitors of a tripeptidyl peptidase II, to reduce centrosome duplication errors that are associated with cancer cells.

### BACKGROUND

Genomic instability is frequently found in tumors caused by overexpression of the c-myc proto-oncogene (McCormack et al., 1998 Oncogene 16, 2755-66). It is believed that genomic instability contributes essentially to the ability of cancer cells to overcome selection barriers during malignant progression (Klausner, 2002 Cancer Cell 1, 3-10) and to the development of chemotherapy resistance (Campain, et al. 1995 Somat Cell Mol Genet 21, 451-71). Numerical and structural chromosomal aberrations are rapidly induced when c-Myc is expressed in normal fibroblasts (Felsher, et al. 1999 Proc Natl Acad Sci USA 96, 3940-4) or other cell types (Fest, et al. 2002 Oncogene 21, 2981-2990). Burkitt's lymphomas frequently show not only the characteristic c-myc-activating (8;14) chromosomal translocation but also subtle numerical chromosome imbalances (Mitelman, et al. 2002 Mitelman database of chromosome aberrations in cancer).

The c-myc proto-oncogene is overexpressed in major human cancers (DePinho et al., 1991 Adv Cancer Res 57, 1-46). c-myc encodes a transcription factor which contributes to tumorigenesis by inducing alterations of cell growth, differentiation, apoptosis (reviewed in Grandori, et al. 2000 Annu Rev Cell Dev Biol 16, 653-99). Overexpression of c-Myc also results in destabilization of the cellular genome (Fest, et al. 2002 Oncogene 21, 2981-2990). Genomic instability is a hallmark of malignant growth and has been proposed to enable tumor cells to overcome selection barriers during malignant progression (Klausner, 2002 Cancer Cell 1, 3-10). Genomic instability plays also an important role for the development of chemotherapy resistance (Campain, et al. 1995 Somat Cell Mol Genet 21, 451-71). However, the mechanisms by which c-Myc elicits genomic instability and potential strategies to suppress this process have not been explored in detail.

Burkitt's lymphoma is a highly aggressive B cell neoplasia characterized by chromosomal translocations which constitutively activate the c-myc proto-oncogene (Boxer, et al. 2001 Oncogene 20, 5595-5610). These translocations involve the c-myc locus on chromosome 8 and typically one of the immunoglobulin loci for example the immunoglobulin heavy chain locus on chromosome 14 (Klein, 1983 Cell 32, 311-5). About 40% of Burkitt's lymphomas show inactivation of the p53 tumor suppressor protein (Bhatia, et al. 1992 Cancer Res 52, 4273-6) and although loss of p53 can promote genomic instability in c-Myc expressing cells (Yin, et al. 1999 Oncogene 18, 1177-84), loss of p53 function is not required for c-Myc to induce genomic destabilization (McCormack, et al. 1998 Oncogene 16, 2755-66).

In addition to chromosomal translocations, Burkitt's lymphoma cells frequently show subtle signs of mitotic infidelity with gains and losses of whole chromosomes (Fest, 2002 Oncogene 21, 2981-2990; Mitelman, et al. 2002 Mitelman database of chromosome aberrations in cancer). This form of chromosomal instability typically results from abnormal multipolar cell divisions. Multipolar mitoses with abnormal numbers of spindle poles can appear after centrosome duplication errors (Pihan, et al. 1999 Sem Cancer Biol. 9, 289-302; Salisbury, et al. 1999 Biol. Cell 91, 451-60). The centrosome is a small cytoplasmic organelle consisting of a pair of centrioles, short cylinders of triplet microtubules, embedded in a pericentriolar protein matrix (Hinchcliffe, et al. 2001 Genes Dev 15, 1167-81; Stearns, 2001 Cell 105, 417-20). Centrosomes are the major microtubule organizing centers in mammalian cells but are also involved in various other processes (Doxsey, 2001 Nat Cell Biol 3, El 05-8). In order to accomplish its function to organize a bipolar mitotic spindle, the single centrosome of a cell duplicates precisely once before mitosis. This process is tightly linked to the cell division cycle through activation of cyclin/cdk2 complexes at the G1/S transition (Hinchcliffe, et al. 1999 Science 283, 851-4; Lacey, et al. 1999 Proc Natl Acad Sci USA 96, 2817-22; Matsumoto, 1999 Curr Biol. 429-32; Meraldi, 1999 Nat Cell Biol 1, 88-93). Failure to control centrosome duplication can give rise to abnormal centrosome numbers, which are frequently detected in various human malignancies (Pihan, 1998 Cancer Res 58, 397465).

In analogy to the G1/S transition of the cell division cycle, initiation of centrosome duplication depends on proteolytic processes mediated by the ubiquitin/proteasome machinery (Freed, 1999 Genes Dev 13, 2242-57). Centrosomes harbor components of the 26S proteasome (Fabunmi, 2000 J Biol Chem 275, 409-13) as well as Skp1-cullin-F-box (SCF) ubiquitin ligase subunits (Freed, 1999 Genes Dev 13, 2242-57; Gstaiger, 1999 Exp Cell Res 247, 554-562). Centrosome duplication depends on a functional ubiquitin/proteasome system (Freed, 1999 Genes Dev 13, 2242-57).

Unless otherwise defined, all technical and scientific terms herein have the same meaning as commonly understood by one of ordinary skill in the art to which this invention pertains. Although methods and materials similar or equivalent to those described herein can be used in the practice of the present invention, suitable methods and materials are described below. All publication, patents, and patent applications mentioned herein are incorporated by reference in their entirety. The examples and claims herein are for illustrative purposes and are not intended to be further limiting.

### SUMMARY

The invention features a method of reducing centrosome duplication errors in a cell, comprising treating the cell with an effective dosage of an inhibitor of a tripeptidyl peptidase II (TPPII). For example, the inhibitor is butabindide, or the inhibitor is a derivative of butabindide. In most embodiments, the cell is a cancer cell. For example, the cancer cell is malignant. For example, the cell shows altered expression or activity of c-myc. Further, the cancer cell contains a centrosome abnormality. The cancer can be any type, for example, lymphoma, leukemia, lung, head and neck, colorectal carcinoma, prostate, breast, skin, melanoma, ovarian, brain, esophageal, gastric, and liver.

In another embodiment, the invention provides a method of inhibiting growth of a lymphoma cell, comprising administering an inhibitor of a tripeptidyl peptidase II to a subject having a lymphoma. The cell can be a lymphomas, for example, a B or T cell lymphoma. Further, the cell can be from a lymphoma such as Hodgkin's, non-Hodgkins, and Burkitt's lymphomas.

In yet another embodiment, the invention provides a method of decreasing viability of a lymphoma cell, by treating the cell with an effective dosage of an inhibitor of a tripeptidyl peptidase II. For example, the inhibitor is a butabindide compound, such as UCL1371 and butabindide. The dosage can be at least about 100 micromolar, for example, about 150 micromolar, about 200 micromolar, about 250 micromolar, or about 500 micromolar.

In another embodiment, the invention provides a method of identifying an antitumor agent, comprising screening for an inhibitor of TPPII. An advantage of this method is that it involves contacting the cell with a TPPII inhibitor. Human cancer cells can be used, and efficacious compounds of necessity permeate the cell. In the method of identifying the antitumor agent, the cancer cell can carry a myc mutation. Further, the cancer cell can be a lymphoma cell. Alternatively, the method involves using an isolated TPPII, or a cell extract. A control for the method is a known TPPII inhibitor.

The TPPII inhibitor is a butabindide compound, which is defined herein to be a derivative of Formula (I).

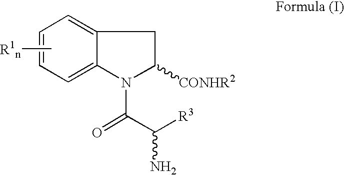

The butabindide compounds includes formula (I) compounds wherein each of the number of n R¹ groups (covalently attached to the 6-membered ring of the indoline moiety) may be the same or different, and is selected from the group consisting of halogen, OH; C₁-C₆ alkyl optionally substituted by one or more radicals selected from the group consisting of halogen and OH; (C₁-C₆) alkenyl optionally substituted by one or more radicals selected from the group consisting of halogen and OH; (C₁-C₆) alkynyl, optionally substituted by one or more radicals selected from the group consisting of halogen and OH, X(C₁-C₆)alkyl, wherein X is S, O, or OCO, and the alkyl is optionally substituted by one or more radicals selected from the group consisting of halogen and OH; SO₂(C₁-C₆)alkyl, optionally substituted by at least one halogen, YSO₃H, YSO₂(C₁-C₆)alkyl, wherein Y is O or NH and the alkyl is optionally substituted by at least one halogen, a diradical —X¹-(C₁-C₆)alkylene-X¹ is O or S; and a benzene ring fused to the indoline ring; n is from 0 to 4; R² (at the carboxyamide end of the molecule) is CH₂R⁴, wherein R⁴ is C₁-C₆ alkyl substituted by one or more radicals selected from the group consisting of halogen and OH; (CH)ₚZ(CH₂)_{q}CH₃, wherein Z is O or S, p is from 0 to 5 and q is from 0 to 5; (C₁-C₆) unsaturated alkyl; or (C₃-C₆) cycloalkyl; or R2 is (C₁-C₆)alkyl or O(C₁-C₆)alkyl, each optionally substituted by at least one halogen; R³ at the amino end of the molecule is H; (C₁-C₆)alkyl optionally substituted by at least one halogen; (CH2)ₚZR⁵ wherein p is from 1 to 3, Z is O or S and R⁵ is H or (C₁-C₆)alkyl; benzyl; or a pharmaceutically acceptable acid addition salt thereof; provided that when R³ is a halogen atom, a O—(C₁-C₆)alkyl; OH or (C₁-C₄)alky group; R² is CH₂R⁴ wherein R⁴ is (CH₂)₂SCH₃, —(CH₂)₂OH or cyclohexyl; or R² is a (C₁-C₆)alkyl group; then R³ is neither a hydrogen atom nor a (C C₄)alkyl group.

Other known inhibitors are AAF-CMK and AAF-MCA, the AAF being tripeptide ala-ala-phe, and derivatives of these inhibitors, which are commercially available.

In another embodiment, the invention provides a method of diagnosing a cancer cell or a precancerous cell, the method comprising identifying a cell having centrosomal abnormalities. For example, the method centrosomal abnormalities comprise the cell having a greater number of centrosomes or mitotic spindle poles than a normal cell. Further, the growth of the cell is inhibited by a TPPII inhibitor, including any of those described above.

Also provided is a method of prognosis of susceptibility of test cell to treatment with a TPPII inhibitor, the method involving determining frequency in the cell of centrosomal abnormalities compared to that of a normal control cell, and is used for the test cell that is in need of diagnosis and prognosis for a cancer or a precancerous condition. The method is determining growth rate of the cell in the presence of the TPPII inhibitor compared to growth rate of the control cell, such that a greater frequency of centrosomal abnormalities and an inhibition of the growth rate by the TPPII inhibitor in the test cell, compared to the normal cell, indicates that the test cell is cancerous or precancerous, and that the cancer can be treated with a TPPII inhibitor.

Also provided is a method of inhibiting progression of cancer in a precancerous cell, comprising contacting the cell with a TPPII inhibitor. For example, the TPPII inhibitor is a butabindide compound, such as butabindide.

Also provided are compositions for use in treating a cancerous or precancerous condition comprising a TPPII inhibitor and an anti-cancer agent. The anti-cancer agent is any known in the art, for example, a cis-platin, a proteosome inhibitor such as Velcade (PS-341), an antibody such as Erbitux, a taxol such as Taxotere, a camptothecan such as Irinotecan, or a Gleevec.

Kits for these compositions and methods are provided, comprising reagents to measure centrosomal abnormalities, a container, and instructions for use. Some kits further include a TPPII inhibitor.

### BRIEF DESCRIPTION OF THE DRAWINGS

FIG. 1 is a set of photomicrographs and a bar graph showing that overexpression of c-Myc induces centrosome abnormalities. FIG. 1a is a photograph of a karyotype of a Burkitt's lymphoma cell from a case revealing the (8;14) chromosomal translocation (arrows). In addition, this case shows loss of one copy of chromosome 17 (arrowhead) and other cytogenetic changes. FIG. 1b is a photograph of hematoxylin and eosin staining of cells from the same Burkitt's lymphoma case. Multiple mitotic figures are present, one with abnormal configuration (arrowhead). FIG. 1c shows visualization of centrosomes in the same case. Arrow indicates normal centrosomes (lower right insert). Arrowheads indicate abnormal centrosomes (other inserts). Abnormalities include aberrant numbers of centrosomes (upper right insert), increase of centrosome size (large dots in upper left and lower left inserts), and structural abnormalities (elongated structure in lower left insert). Immunofluorescence for γ-tubulin is from rhodamine red. Nuclei are stained with DAPI (4′,6′-diamidino-2-phenylindole hydrochloride). FIG. 1d is an immunoblot analysis of U20S cells transiently transfected with c-myc or empty vector (neo). Actin was used as loading control. FIG. 1e shows that transient transfection of normal human keratinocytes (NHKs) with c-myc results in abnormal centrosome numbers 48 hours post transfection (right panel; arrowhead, insert). NHK transfected with empty vector display normal centrosomes (left panel; arrowhead, insert). Immunofluorescence for γ-tubulin is shown with FITC, Mitochondrial DsRed is the transfection marker, and nuclei were stained with DAPI. FIG. 1f is a bar graph that shows quantitation of cells with abnormal centrosome numbers in NHKs transient transfected with c-myc or empty vectors (neo). Each error bar indicates an average+standard error of at least three independent experiments. FIG. 1g is an analysis of chromosome 11 copy numbers in NHKs transiently transfected with c-myc (right panel) or controls (left panel). Fluorescence in situ hybridzation for chromosome 11 shows green dots, and nuclei were stained with DAPI. Scale bars indicate a length of 10 μm.

FIG. 2 is a set of photographs of polyacrylamide gel bands, cells, and bar graphs showing that overexpression of c-Myc induces centrosome and centriole duplication errors. FIG. 2a is an immunoblot analysis of U20S cells transiently transfected with empty vector (neo), or plasmids encoding c-Myc, HPV-16 E6, HPV-16 E7, or E2F-1. Expression of HPV-16 E6 was ascertained by reduced p53 protein levels. FIG. 2b shows abnormal centrosome numbers in a U20S cell transiently transfected with c-myc (right panel; arrowhead, insert). Normal centrosomes in a cell transfected with control vector (left panel; arrowhead, insert). Immunofluorescence for γ-tubulin is shown with rhodamine red. Farnesylatable GFP is used as the transfection marker. Nuclei were stained with DAPI. FIG. 2c shows quantitation of the proportion of cells with abnormal centrosome numbers in U20S cells transiently transfected with increasing amounts of c-myc. Each bar indicates average+standard error of at least three independent experiments. FIG. 2d shows abnormal numbers on centrioles in U20S cells stably expressing GFP-tagged centrin (U20S/centrin-GFP) and transiently transfected with c-myc (right panel; arrowhead, insert). Normal centrioles in a U20S/centrin-GFP cells transiently transfected with empty vector (left panel; arrowheads, insert). Mitochondrial DsRed was used as transfection control. Nuclei were stained with DAPI. FIG. 2e shows abnormal tripolar prometaphase in a U20S/centrin-GFP cell transiently transfected with c-myc (right panel, arrowheads). Normal metaphase is shown with two centrioles at each spindle pole. Mitochodrial DsRed was used as the transfection marker. Chromosomes were stained with DAPI. Scale bars indicate 10 μm. FIG. 2f shows quantitation of abnormal centriole numbers in U20S/centrin-GFP cells expressing the indicated proteins after transient transfection. Each bar indicates the average+standard error of at least three independent experiments.

FIG. 3 is a set of photographs of acrylamide gel protein bands and bar graphs that show that c-Myc induced centriole duplication errors are cell cycle dependent but are less sensitive to proteasome inhibition compared to other oncogenic stimuli. FIG. 3a is an Immunoblot analysis of U20S/centrin-GFP cells that were transfected with c-Myc, E2F-1, or HPV-16 E7 and were cotransfected with the indicated amounts of HA-tagged dominant-negative mutant DP1 (DN-DP1). FIG. 3b shows quantitation of the proportion of cells with abnormal centriole numbers in U20S/centrin-GFP cells 48 hours after co-transfection of c-Myc, E2F-1, and HPV-16 E7, respectively, with the indicated amount of DN-DP 1-HA encoding plasmids. In all experiments, only transfected cells expressing DsRed as transfection marker were evaluated. FIG. 3c shows quantitation of centriole abnormalities in U20S/centrin-GFP cells transiently transfected with empty vector (neo), or c-Myc, E2F-1, and HPV-16 E7, respectively, after treatment with the proteasome inhibitor clastolactacystin β-lactone (CLBL), or DMSO as solvent control. In all experiments, only transfected cells expressing DsRed as transfection marker were evaluated. Each bar indicates average+standard error of at least three independent experiments.

FIG. 4 is a photograph of an acrylamide gel, a set of bar graphs and DNA content analyses that show that c-Myc induced centriole duplication errors are abrogated by inhibitors of tripeptidyl peptidase II (TPPII). FIG. 4a is an immunoblot analysis of TPPII expression in U20S cells transiently transfected with empty vector (neo) or c-myc, E2F-1, or HPV-16 E7 encoding plasmids. Actin was used as loading control. FIG. 4b is a quantitation of U20S/centrin-GFP cells with abnormal centriole numbers after transient transfection with empty vector (neo), c-Myc, E2F-1, or HPV-16, and treatment with 1 μM or 10 μM AAF-CMK or DMSO used as solvent control, respectively. Each bar indicates average+standard error of at least three independent experiments. FIG. 4c is a quantitation of U20S/centrin-GFP cells with abnormal centriole numbers after transient transfection of c-Myc and treatment with increasing concentrations of Butabindide. Each bar indicates average+standard error of at least three independent experiments. FIG. 4d is an analysis of DNA content of U20S/centrin-GFP cells either untreated or treated for 24 hours with 1% DMSO, 10 μM AAF-CMK, or 10 μM Butabindide.

FIG. 5 is a set of line graphs, photographs, and a bar graph that show that adapted Burkitt's lymphoma cells with upregulated TPPII expression are growth-inhibited by Butabindide. FIG. 5a is an analysis of viability of GA-10 cells grown in the presence of 1 μM CLBL or solvent control (0.1% DMSO) as determined by trypan blue exclusion. FIG. 5b is an immunoblot analysis of TPPII and c-Myc expression in GA-10 cells adapted to grow in the presence of 1 μM CLBL or 0.1% DMSO. FIG. 5c is a visualizations of centrosomes in GA-10 cells adapted to grow in the presence of 0.1% DMSO (c) or 1 μM CLBL right. Immunofluorescence for γ-tubulin is rhodamine red. Nuclei were stained with DAPI. FIG. 5d is a set of graphs showing inhibition of numbers of viable cells as a function of time of culture, in days, with various concentrations (0.1, 1.0, 10, 100 or 500 μM) if Butabindide, for GA-10 cells (left), and GA-10 cells adapted to group in the presence of 1 μM CLBL (right). FIGS. 5e and 5f are photographs of results of a soft agar colony formation test of GA-10 cells adapted to grow in the presence of 1 μM CLBL, and treated with 10 μM Butabindide (right) or solvent control (left) for one week. FIG. 5g is a bar graph showing quantitation of the soft agar colony formation of GA-10 cells adapted to grow in the presence of 1 μM CLBL and treated with 10 μM Butabindide or solvent control (untreated) for one week. Each bar indicates average+standard error of triplicate colony counts. FIG. 5h shows photomicrographs of centrosomes (arrows) attached to mitotic spindles (left) or unable to form a spindle (right). The frequency of cells showing inhibition of spindle formation was increased from 44% to 66% of the total in the presence of Butabindide (right).

### DETAILED DESCRIPTION OF SPECIFIC EMBODIMENTS

The proteasome machinery has been shown to be functionally impaired in Burkitt's lymphoma cells (Frisan, et al. 2000 Int J Cancer 88, 681-8). This defect is compensated by an upregulation of enzymes involved in alternative proteolytic pathways, for example, the cytoplasmic oligopeptidase tripeptidyl peptidase II (TPPII; Gavioli, et al. 2001 Nat Cell Biol. 3, 283-8) which allows at least a partially continued ubiquitin-dependent proteolysis (Wang, et al. 2000 Proc Natl. Acad. Sci. USA 97, 9990-5).

Cancer detection by centrosome abnormality is shown in U.S. Pat. No. 5,972,62. It is demonstrated herein that high levels of centrosome abnormalities are found in a human Burkitt's lymphoma harboring the characteristic (8; 14) chromosomal translocation. Overexpression of c-Myc rapidly induces centrosome and centriole duplication errors. This novel oncogenic activity of c-Myc involves its ability to disrupt regulatory nodes that govern both cell cycle progression and centrosome duplication. Most strikingly, however, c-Myc induced abnormal centrosome duplication shows weak sensitivity against proteasome inhibition but is efficiently and selectively abrogated using the tripepidyl peptidase II (TPPII) inhibitors AAF-CMK and butabindide. This demonstrates that upregulation of the oligopeptidase TPPII is not only important for cell survival upon impaired proteasome activity in c-Myc overexpressing cells (Wang, 2000 Proc Natl Acad Sci USA 97, 9990-9995) but is also required to maintain complex regulatory cellular processes including centrosome duplication. As a consequence, Burkitt's lymphoma cells adapted to grow in the presence of a proteasome inhibitor upregulate TPPII protein levels and maintain levels of centrosome abnormalities similar to controls.

Discovery of new anti-cancer agents remains an important pharmacological activity. An inhibitor of TPPII might selectively inhibit Burkitt's lymphoma cells and other cancer cells. It is shown in the Examples herein that growth and soft agar colony formation of adapted cells are significantly inhibited by butabindide. Pharmacological inhibition of TPPII activity using potent and selective inhibitors, such as butabindide, represents a promising treatment strategy to target centrosome-related mitotic infidelity and genomic instability. This approach may help to suppress malignant progression and chemotherapy resistance in tumors with overexpression of c-Myc.

As used herein, “butabindide compound” shall mean one of a group of a compounds that are shown in U.S. Pat. No. 6,335,360, the butabindide compounds all being related to formula (I) in that patent which is hereby incorporated by reference herein. Methods for synthesis of these compounds are shown in the examples section of 6,335,360. Derivatives of butabindide which comprise butabindide compounds include formula (I) compounds wherein each of the n R¹ groups (covalently attached to the 6membered ring of the indoline moiety) may be the same or different, and are selected from the group consisting of halogen, OH; C₁-C₆ alkyl optionally substituted by one or more radicals selected from the group consisting of halogen and OH; (C₁-C₆) alkenyl optionally substituted by one or more radicals selected from the group consisting of halogen and OH; (C₁-C₆) alkynyl, optionally substituted by one or more radicals selected from the group consisting of halogen and OH, X(C₁-C₆)alkyl, wherein X is S, O, or OCO, and the alkyl is optionally substituted by one or more radicals selected from the group consisting of halogen and OH; SO₂(C₁-C₆)alkyl, optionally substituted by at least one halogen, YSO₃H, YSO₂(C₁-C₆)alkyl, wherein Y is O or NH and the alkyl is optionally substituted by at least one halogen, a diradical —X¹—(C₁-C₆)alkylene-X¹ is O or S; and a benzene ring fused to the indoline ring; n is from 0 to 4; R² (at the carboxyamide end of the molecule) is CH₂R⁴, wherein R⁴ is C₁-C₆ alkyl substituted by one or more radicals selected from the group consisting of halogen and OH; (CH)ₚZ(CH₂)_{q}CH₃, wherein Z is O or S, p is from 0 to 5 and q is from 0 to 5; (C₁-C₆) unsaturated alkyl; or (C₃-C₆) cycloalkyl; or R2 is (C₁-C₆)alkyl or O(C₁-C₆)alkyl, each optionally substituted by at least one halogen; R³ at the amino end of the molecule is H; (C₁-C₆)alkyl optionally substituted by at least one halogen; (CH2)ₚZR⁵ wherein p is from 1 to 3, Z is O or S and R⁵ is H or (C₁-C₆)alkyl; benzyl; or a pharmaceutically acceptable acid addition salt thereof; provided that when R³ is a halogen atom, a O—(C₁-C₆)alkyl; OH or (C₁-C₄)alky group; R² is CH₂R⁴ wherein R⁴ is (CH₂)₂SCH₃, —(CH₂)₂OH or cyclohexyl; or R² is a (C₁-C₆)alkyl group; then R³ is neither a hydrogen atom nor a (C₁-C₄)alkyl group.

A butabindide compound which is closely related to butabindide is UCL 1371 (Rose et al., 1996 Nature 380: 403-409), having a Kᵢ for TPPII of 80 nM, rather than a Kᵢ for TPPII of 7 nM as was determined for butabindide. These two compounds are structurally similar, being identical except for the fused indoline two ring structure, of butabindide, compared to the single 5-membered ring structure of UCL 1371. The differences in inhibitory ability reflect the details of the structural relationship of the inhibitor in the active site of the TPPII enzyme. These and other butabindide compounds are tested herein for optimal properties as anti-tumor agents.

A pharmaceutically acceptable carrier includes any and all solvents, dispersion media, coatings, antimicrobials such as antibacterial and antifungal agents, isotonic and absorption delaying agents and the like that are physiologically compatible. Preferably, the carrier is suitable for oral, intravenous, intramuscular, intraperitoneal, transdermal, or subcutaneous administration, and the active compound can be coated in a material to protect it from inactivation by the action of acids or other adverse natural conditions.

A composition of the present invention can be administered by a variety of methods known in the art as will be appreciated by the skilled artisan. The active compound can be prepared with carriers that will protect it against rapid release, such as a controlled release formulation, including implants, transdermal patches, microencapsulated delivery systems. Many methods for the preparation of such formulations are patented and are generally known to those skilled in the art. See, e.g., Sustained and Controlled Release Drug Delivery Systems, J. R. Robinson, Ed. Marcel Dekker, Inc., NY (1978).

Therapeutic compositions for delivery in a pharmaceutically acceptable carrier are sterile, and are preferably stable under the conditions of manufacture and storage. The composition can be formulated as a solution, microemulsion, liposome, or other ordered structure suitable to high drug concentration. Dosage regimens can be adjusted to provide the optimum desired response (e.g., a therapeutic response). For example, a single bolus can be administered, several divided doses can be administered over time, or the dose can be proportionally reduced or increased as indicated by the exigencies of the disease situation.

In general, a preferred embodiment of the invention is to administer a suitable daily dose of a butabindide composition that will be the lowest effective dose to produce a therapeutic effect, for example, mitigation of symptoms such as inhibiting growth of a tumor or causing regression in size of the tumor. The therapeutic compounds of the invention are preferably administered at a dose per subject per day of at least 2 mg, at least 5 mg, at least 10 mg or at least 20 mg as appropriate minimal starting dosages. In general, the compound of the effective dose of the composition of the invention can be administered in the range of 50 to 400 micrograms of the compound per kilogram of the subject per day. Alternatively, an effective dose of the therapeutic compounds is at least an in vivo concentration of about 0.1 μM, about 1.0 μM, about 10 μM, about 100 μM, or about 500 μM.

A physician or veterinarian having ordinary skill in the art can readily determine and prescribe the effective dose of the pharmaceutical composition required. For example, the physician or veterinarian could start doses of the compound of the invention employed in the pharmaceutical composition at a level lower than that required in order to achieve the desired therapeutic effect, and increase the dosage with time until the desired effect is achieved.

In another preferred embodiment, the pharmaceutical composition includes also an additional therapeutic agent. Thus in a method of the invention, the pharmaceutical composition can be administered as part of a combination therapy, i.e. in combination with an additional agent or agents. Examples of materials that can be used as combination therapeutics with the present butabindide compounds for treatment of tumors and cancer conditions as additional therapeutic agents include: an antibody or an antibody fragment that can bind specifically to a protein on a cancer cell such as HER-2 or CEA; a bispecific antibody capable of binding to a cancer call and effecting lysis by a macrophage; a chemotherapeutic agent such as 5-fluorouracil, methotrexate, paclitaxel, suramin, cisplatin, or adriamycin; a growth inhibitory peptide; an inhibitor of neovascularization, i.e., an anti-angiogenesis agent, for example, a protein such as endostatin or angiostatin; or an anti-microbial agent such as an antibiotic, an antifungal agent, or an antiviral agent.

An improvement in the symptoms as a result of such administration is noted by a reduction in tumor size or disappearance of the tumor; or reduction in appearance or growth of tumors. A therapeutically effective dosage preferably reduces tumor growth or metastasis by at least about 20%, more preferably by at least about 40%, even more preferably by at least about 60%, and even still more preferably by at least about 80%, relative to untreated subjects.

Embodiments of the invention are fully described, and are further illustrated by the following examples, which are included for illustrative purposes and are not to be construed as limiting.

### EXAMPLES

The following Methods and Materials were used throughout the Examples.

Cell Culture and Transfections. Normal human keratinocytes (NHKs) were isolated from neonatal foreskins and cultured as described previously (Jones, et al. 1997 Genes Dev 11, 2101-11). NHKs were transiently transfected using FuGENE 6 (Roche Molecular Biochemicals, Indianapolis, Ind.) according to manufacturer's instructions. The human osteosarcoma cell line U20S was obtained from the American Type Culture Collection (ATCC) and maintained in Dulbecco's modified Eagle medium (DMEM) supplemented with 10% fetal bovine serum, 50 units/ml penicillin and 50 μg/ml streptomycin. Stable populations expressing centrin-GFP were generated by transfecting U20S cells with 10 μg of a centrin-GFP construct using calcium phosphate co-precipitation (Chen, et al. 1987 Mol Cell Biol 7, 2745-2752) and expansion of stable clones after G418 selection. All transient transfection experiments in U20S/centrin-GFP cells were performed using calcium phosphate coprecipitation (Chen, 1987 Mol Cell Biol 7, 2745-2752). Expression plasmids used were c-Myc, E2F-1, HPV-16 E7, HPV-16 E6, or a hemagglutinin-tagged dominant-negative DP1 mutant (DP 1-HA). Empty vector controls (pCMVneo) were included in all experiments. In all transient transfection experiments, cells were co-transfected with either farnesylatable green fluorescent protein (GFP), DsRed fluorescent protein, or cyan fluorescent protein (CFP; all from Clontech, Inc., Palo Alto, Calif.) as indicated and only cells expressing the respective fluorescent protein were evaluated. The human Burkitt's lymphoma cell line GA-10 was obtained from ATCC and maintained in RPMI 1640 medium containing 10 mM HEPES and supplemented with 10% fetal bovine serum, 50 units/ml penicillin and 50 μg/ml streptomycin. Adapted GA-10 cells were obtained by cultivation of cells in the presence of 1 μM clastolactacystin β-lactone (CLBL; Sigma Corp., St. Louis, Mo.) or 0.1% dimethyl sulfoxide (DMSO; ICN, Inc., Costa Mesa, California).

Immunological Methods. Immunoblot analysis was performed as described previously (Jones et al., 1997). Antibodies used were directed against c-Myc (9E10; Covance/Babco, Berkeley, California), p53 (Ab-6; Oncogene Research Products, Inc., San Diego, Calif.), HPV-16 E7 (ED17; Santa Cruz Biotechnology, Inc., Santa Cruz, California), E2F-1 (C-20; Santa Cruz), HA (12CA5; Boehringer Mannheim, Mannheim, Germany), tripetidyl peptidase II (OEM Concepts, Inc.; Toms River, New Jersey), or Actin (C-2, Santa Cruz).

For immunofluorescence analysis, sections of formalin-fixed, paraffin-embedded tissue were processed as described previously (Duensing, 2000 Proc Natl Acad 801 USA 97, 10002-7). Cultured cells were either grown on coverslips or cytospin preparations were made from cells growing in suspension. Cells were fixed in 4% paraformaldehyde in PBS and permeabilized with 1% Triton-X 100 in PBS for 10 min each at room temperature. Cells were then blocked with 10% normal donkey serum (Jackson Immunoresearch, West Grove, Pa.) and incubated with a mouse monoclonal anti-γ-tubulin antibody (GTU-88; Sigma Corp.; St. Louis, Mo.) at a 1:2000 dilution overnight at 4° C. followed by a donkey anti-mouse rhodamine red conjugated secondary antibody (Jackson ImmunoResearch Laboratories, Inc., West Grove, Pa.) at a 1:100 dilution in PBS or a donkey anti-mouse FITC labeled secondary antibody at a 1:200 dilution in PBS for 2 hours at 37° C. Ki-67 was detected using a mouse monoclonal antibody (clone Ki-67; Dako Cytomation California, Inc., Carpinteria, California) at a 1:100 dilution followed by a rhodomine red-labeled donkey anti-mouse secondary antibody as described above. TPPII and γ-tubulin co-staining was performed by incubation of cells with chicken anti-TPPII (OEM) at a 1:500 dilution overnight at 4° C. followed by a rhodamine red-labeled donkey anti-chicken secondary antibody at a 1:100 dilution in PBS for 2 hours at 37° C., then followed by a anti-γ-tubulin antibody and a donkey anti-mouse FITC labeled secondary antibody as described above. Cells were finally washed in PBS and counterstained with 4′,6′-diamidino-2-phenylindole (DAPI; Vector, Burlingame, Calif.). Cells were analyzed using a Leica DMLB epifluorescence microscope equipped with a multiband and a CFP filter set (Omega Optical, Brattleboro, Vt.) and a Sony DKC5000 camera system. Pictures were transferred to Adobe Photoshop for printout.

Proteasome and TPPII inhibition. U20S/centrin-GFP cells were transiently transfected with expression plasmids as indicated using calcium phosphate coprecipitation, washed with PBS 14 hours after transfection, incubated in normal growth media for 8 hours, and incubated in the presence of the inhibitor for another 24 hours. For proteasome inhibition, clastolactacystin β-lactone (CLBL; Sigma Corp.) was used at a 0.8 mM concentration dissolved in DMSO. The TPPII inhibitor H-Ala-Ala-Phe-chloromethylketone (AAF-CMK; Sigma Corp.) was used at a 1 or 10 μM concentration, respectively, dissolved in DMSO. TPPII inhibitors such as AAF-MCA (ala-ala-phe-methylcoumarin amide) are available from Peptides International, Inc., Louisville, Ky. Butabindide (Tocris Cookson, Inc., Bristol, United Kingdom) was diluted in sterile water and used at the concentrations indicated. Solvent controls (DMSO or water) were included in all experiments.

Clonogenic assay. For assessment of soft agar colony formation, adapted GA-10 cells were suspended in 37° C. RPMI 1640 medium containing 0.3% bacto-agar (Difco Corp., Detroit, Mich.), 10 mM HEPES, 20% fetal bovine serum, 50 units/ml penicillin, 50 μg/ml streptomycin, and 10 μM butabindide or sterile water as solvent control. The agar was allowed to set for 1 hour at room temperature, plates were then overlaid with growth media with or without butabindide, transferred to 37° C. and colony growth was assessed after one week.

Tumor cytogenetics. GTG-banded metaphase cells were obtained from unstimulated 24 cultures and routine karyotyping was performed.

Fluorescence in situ hybridization (FISH). For interphase in situ hybridization analysis, a Spectrum Green-labeled chromosome 11 α-satellite probe (D11Z1) was used according to manufacturer's protocol (Vysis, Inc., Downers Grove, Ill.). Cells were counterstained with DAPI (Vector).

Flow cytometry. DNA content of cells was analyzed by propidium iodide staining followed by flow cytometry as described previously (Thompson, et al. 1997 Oncogene 15, 3025-35).

Statistical Analysis. At least three independent experiments were performed if not indicated otherwise. Mean and standard error are given. Statistical significance was assessed using the two-tailed Student's t test for independent samples.

### Example 1

### Overexpression of c-Myc Induces Centrosome Duplication Errors and Mitotic Infidelity

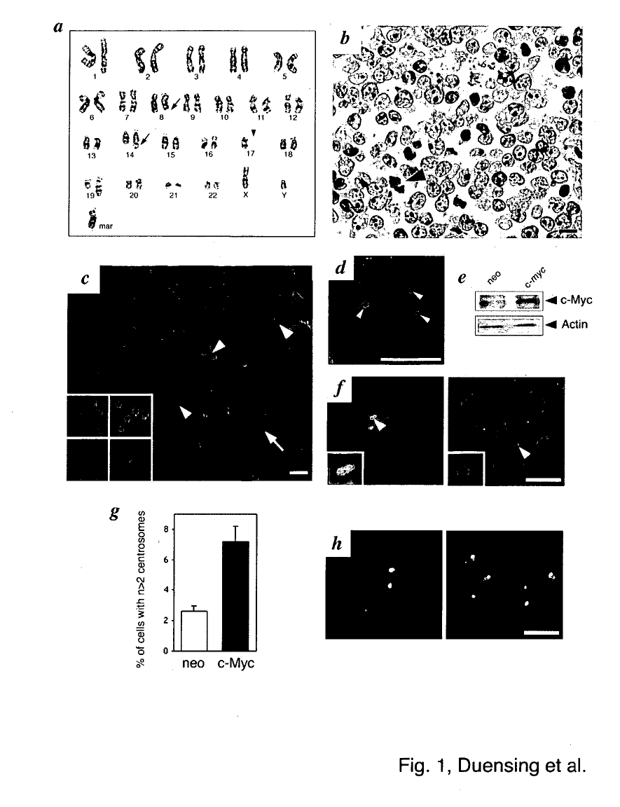

Chromosomal instability with gains or losses of whole chromosomes can result from centrosome-related mitotic disturbances (Pihan, et al. 1999 Sem Cancer Biol. 9, 289-302 Salisbury, et al. 1999 Biol Cell 91, 451-60). To study the role of c-Myc for the induction of abnormal centrosome numbers and genomic instability, a case of human Burkitt's lymphoma harboring the t(8;14) chromosomal translocation was analyzed (FIG. 1a). In addition to this translocation which results in c-myc activation, conventional karyotyping revealed loss of one copy of chromosome 17 which harbors the p53 tumor suppressor locus on 17p. The complete karyoptype was 46, XY, t(8;14) (q24;q32),-17,der(19)ₜ(7; 19)(q 11.2;q13),+mar (FIG. 1a). Hematoxylin and eosin staining of cells from this case showed the typical morphology of Burkitt's lymphoma and abundant mitotic figures, some of them with abnormal configuration (FIG. 1b).

Since mitotic infidelity and losses of whole chromosomes such as chromosome 17 in the present case can arise from centrosome-associated mitotic defects, whether centrosome abnormalities were present in this case of lymphoma was determined. Since the normal centrosome duplication cycle results in two centrosomes prior to entry into mitosis, cells with more than two centrosomes were considered abnormal. Using immunofluorescence for γ-tubulin, a pericentriolar marker (Steams, 1991 Cell 65, 82536, various numerical as well as structural centrosome abnormalities were detected in up to approximately 30% of cells (FIG. 1c). Besides numerical centrosome abnormalities (FIG. 1c, upper right insert), structural changes were readily observed with increase of centrosome size and/or abnormal shape (exemplified in FIG. 1c, lower left insert).

To test directly whether overexpression of c-Myc can cause centrosome abnormalities, primary human keratinocytes were transiently transfected with a c-myc encoding plasmid (FIG. 1d-f). c-Myc expressing cells were selected, based on use of mitochondrial DsRed expression as a transfection marker. Centrosomes were visualized by immunofluorescence for γ-tubulin (FIG. 1e).

Transient overexpression of c-Myc in primary human keratinocytes resulted in a rapid and significant (p≦0.05) 2.8-fold increase of cells with abnormal centrosome numbers from 2.6% in controls to 7.2% in c-Myc expressing cells 48 hours post transfection (FIG. 1f). In addition, primary human keratinocytes transiently transfected with c-Myc were tested for mitotic infidelity. Fluorescence in situ hybridization (FISH) was used for the α-satellite centromeric region of chromosome 11, to assess chromosome 11 copy numbers in cells. A 7.5% increase in percent of cells with monosomy, trisomy or aneusomy for chromosome 11 was observed in the c-myc transfected population (84 out of 1084; 7.7%) compared to in control cells (37 out of 843; 4.4%) in control cells (FIG. 1g).

These findings show that overexpression of c-Myc rapidly induces abnormal centrosome numbers, and increases the risk for chromosome missegregation in primary human cells. Chromosomal changes can involve losses of chromosome 17 which harbors the p53 tumor suppressor locus (FIG. 1a). Loss of p53 has been reported to cooperate with the c-myc proto-oncogene to induce the formation of certain tumor types (Elson, et al. 1995 Oncogene 11, 181-90). Burkitt's lymphomas generally show a high proliferative activity and can display abnormal mitotic figures (FIG. 1b) which can be the consequence of multiple spindle poles (Duensing, et al. 2000 Proc Natl. Acad. 801 USA 97, 10002-7). Burkitt's lymphoma is here shown to display high levels of centrosome abnormalities including numerical and structural changes. c-Myc is further shown to rapidly induce centrosome duplication errors in primary human cells thereby increasing the risk for mitotic infidelity and chromosome missegregation (FIG. 1g). Abnormal numbers of centrosomes have been reported in many tumor types (Pihan, et al. 1999 Sem Cancer Biol 9, 289-302), however, it is not known whether centrosome abnormalities are the consequence of a primary duplication defect, or whether those abnormalities merely accumulate in neoplastic cells with other abnormalities for example during cytokinesis (Meraldi, et al. 2002 Embo J 21, 483-92).

Evidence herein shows that induction of abnormal centrosome numbers by c-Myc involves a primary centrosome duplication error. This includes the finding that c-Myc induces anormal centrosome numbers in primary human keratinocytes within 48 hours post transfection and that keratinocytes displaying numerical centrosome abnormalities can be mononucleated and morphologically inconspicuous (as exemplified in FIG. 1e). In contrast, cells that become polycentrosomal by accumulation of centrosomes are frequently multinucleated (Meraldi, et al. 2002 Embo J 21, 483-92).

### Example 2

### c-Myc Induced Centrosome Duplication Errors are Associated with Excessive Centriole Duplication

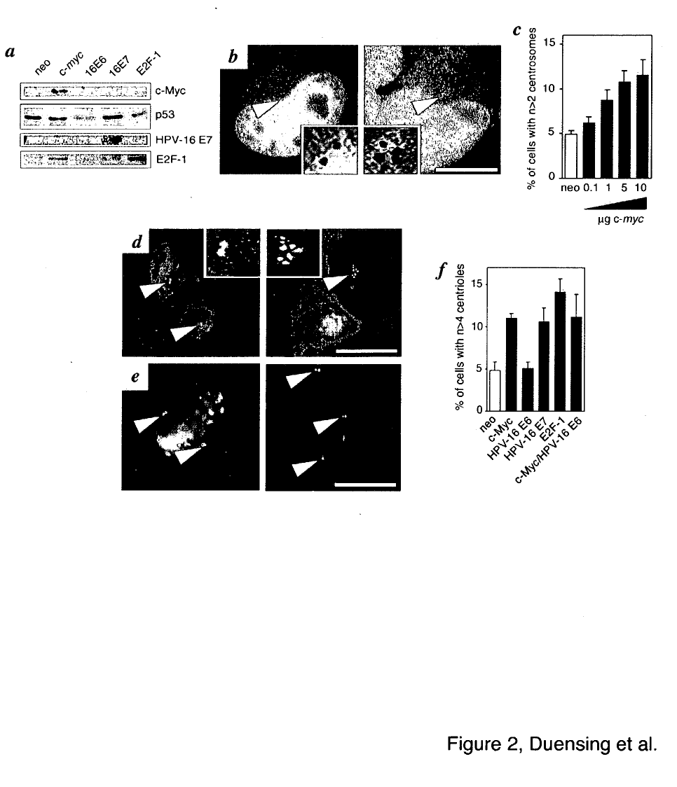

To determine whether the aberrant centrosome numbers generated by c-Myc overexpression are caused by a primary centrosome duplication defect, as opposed to an accumulation of centrosomes induced by defects unrelated to the centrosome duplication cycle (Meraldi, et al. 2002 Embo J 21, 483-92), an association of primary centrosome duplication error in c-Myc expressing cells with an aberrant synthesis of centrioles, the core forming units of the centrosome (Marshall, 2001 Curr Biol 11, R487-96), was analyzed. Since normal human keratinocytes have only a limited number of population doublings in culture, centriole numbers and duplication status in the U20S osteosarcoma cell line engineered to stably express GFP-labeled centrin were assessed (FIG. 2). Centrin is a 20 kD protein that concentrates within the distal lumen of the centrioles (Paoletti, et al. 1996 J. Cell Sci 109, 3089-102), and serves as a marker for individual centrioles (Piel, et al. 2000 J Cell Biol 149, 317-30). In all transient transfection experiments, protein expression was monitored by immunoblot analysis (FIG. 2a).

To test first whether the U20S cell line is a suitable system for centrosome duplication studies, cells were contacted with increasing amounts of c-Myc encoded by DNA, i.e. cells were transiently transfected, and centrosome numbers in the transfectants were analyzed by immunofluorescence microscopy for γ-tubulin (FIGS. 2b,c). A significant (p<0.005) and dose-dependent increase in the proportion of cells with abnormal centrosome numbers was observed, from a baseline level of 4.9% in cells transfected with empty vector, up to 11.5% of cells transfected with 10 μg of c-myc (FIG. 2c).

Whether this observed increase of cells with abnormal centrosome numbers results from a primary duplication error was further addressed by analyzing the ability of c-Myc to induce abnormal centriole synthesis under transient conditions. Centriole number abnormalities induced by c-Myc were compared to those obtained with different oncogenic stimuli which have been reported previously to deregulate centrosome duplication (Duensing, 2000 Proc Natl Acad USA 97, 10002-7). Besides c-Myc, each of HPV-16 E7, the cooperating oncogene HPV-16 E6, and E2F-1 were included in these experiments. U20S/centrin-GFP cells were transiently transfected with expression plasmids as indicated, and mitochondrial DsRed fluorescent protein was used as a transfection marker (FIGS. 2c,d). Since duplication of centrioles prior to a cell division gives rise to four centrioles, cells with more than four centrioles were considered to have an abnormal number of centrioles.

To show that supernumerary centrin-GFP dots are functional centrioles, transfected cells undergoing mitoses were reexamined with GFP-labeled centrioles participating in spindle pole formation (FIG. 2e). Transient transfection of U20S/centrin-GFP cells induced a rapid and significant 2.3-fold (p≦0.05) increase in the proportion of cells with abnormal centriole numbers at a point 48 hours after transfection with c-Myc (11%), compared to controls (4.8%). Transient expression of HPV16 E7, but not HPV16 E6, induced a statistically significant (p≦0.05) increase in the proportion of cells with abnormal centriole numbers (FIG. 2f). Moreover, overexpression of E2F-1 was found to induce a significant (2.9-fold) increase in the proportion of cells with abnormal centriole numbers (14.1%; p≦0.05).

Since overexpression of c-Myc can lead to a p53-dependent G2 arrest in normal fibroblasts (Felsher, et al. 2000 Proc Natl Acad Sci USA 97, 10544-8), whether co-transfection of c-Myc with HPV-16 E6, which diminishes p53 protein levels, can interfere with the number of cells with an abnormal number of centrioles was also tested. No differences were observed between cells co-expressing c-Myc and HPV-16 E6, compared to cells expressing c-Myc alone (FIG. 2f). These findings, together with the observation that c-Myc overexpressing cells do not show an increased proportion of cells with four centrioles characteristic for G2, suggest that, in the present experimental system, a G2 arrest does not play a major role in the observed phenotype.

In summary, data herein demonstrate that overexpression of c-Myc rapidly induced centrosome duplication errors with an excessive duplication of centrioles, the core structures of centrosomes (FIG. 2d).

c-Myc can thus function as a driving force for centrosome duplication errors by inducing excessive centriole duplication. c-Myc as well as HPV-16 E7 and E2F-1 disrupt regulatory nodes that control both cell cycle progression and initiation of the centrosome duplication cycle. c-Myc and HPV-16 E7 have been shown to deregulate the G1/S transition of the cell division cycle leading to an inappropriate S phase entry by activation of cyclin/cdk2 complexes (Amati, et al. 1998 Front Biosci 3, D250-68). Moreover, c-Myc induces the transcriptional activity of E2F transcription factors (Leone, et al. 1997 Nature 387, 422-6). Cyclin/cdk2 activity and E2F-induced gene transcription have been shown to be required for centrosome duplication (Meraldi, 1999 Nat Cell Biol. 1, 88-93), thus blocking cell cycle progression using dominant-negative DPI (Wu, et al. 1996 Mol Cell Biol 16, 3698-706) also abrogates c-myc and E2F-1 or HPV-16 E7 induced centriole amplification.

### Example 3

### c-Myc can Induce Centrosome Duplication Errors by Disrupting Regulatory Nodes that Govern Both Cell Cycle Progression and Centrosome Duplication

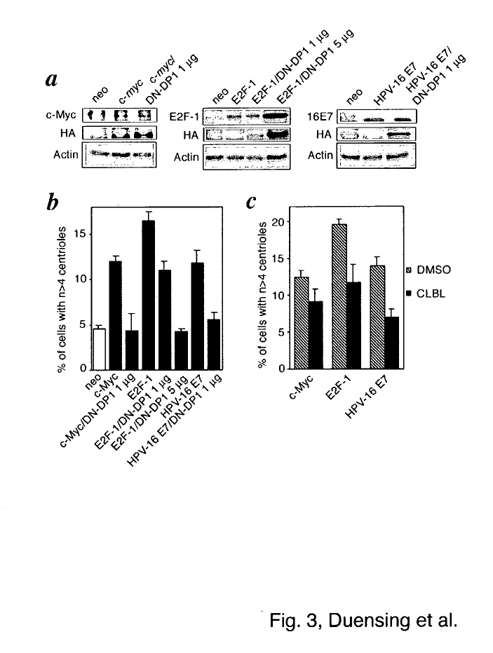

In order to identify the underlying pathways of c-Myc induced abnormal centriole duplication, mechanistic differences between c-Myc and other, functionally and structurally distinct oncogenic stimuli were examined (FIG. 3). The centrosome duplication cycle is closely associated with the cell division cycle. Activation of cyclin/cdk2 complexes in late G1 is required not only for the G1/S transition, but also for the initiation of centrosome duplication (Hinchcliffe, et al. 1999 Science 283, 851-4; Matsumoto, et al. 1999 Curr Biol. 429-32; Meraldi, et al. 1999 Nat Cell Biol 1, 88-93; Lacey, et al. 1999 Proc Natl Acad Sci USA 96, 2817-22). c-Myc can induce unscheduled cdk2 activation through multiple mechanisms (Henriksson, et al. 1996 Adv Cancer Res 68, 109-82). Therefore dependence of ability of c-Myc to induce abnormal centriole numbers on an unrestricted cell cycle progression was analyzed. A dominant-negative mutant of DPI (DN-DP1), a heterodimerization partner required for E2F-induced gene transcription, was used to block cell cycle progression (Wu, et al. 1996 Mol Cell Biol 16, 3698-706).

Co-transfection of HA-tagged DN-DP1 was found to abrogate c-Myc-mediated induction of abnormal centriole numbers in U20S/centrin-GFP cells (FIGS. 3a,b). A similar reduction of cells with abnormal centriole numbers was seen when DN-DP1 was co-expressed with E2F-1 and HPV-16 E7 (FIG. 3b).

In conclusion, c-Myc was found to share ability to induce excessive centriole duplication with other oncogenic stimuli that disrupt control of the G1/S transition of the cell cycle. Further, suppression of E2F-mediated gene transcription also effectively suppressed abnormal centriole duplication.

### Example 4

### c-Myc Induced Abnormal Centriole Duplication is Less Sensitive to Proteasome Inhibition than other Oncogenic Stimuli

Analogous to cell cycle progression, centrosome duplication requires activity of the ubiquitin/proteasome machinery (Freed, et al. 1999 Gene Dev 13, 2242-57). Requirement of the proteasome system for c-Myc-, E2F-1, and HPV-16 E7-induced centriole abnormalities was therefore tested, using the proteasome inhibitor clastolactacystin-β-lactone (CLBL). U20S/centrin-GFP cells were transiently transfected with plasmids as indicated in FIG. 3c, and cells were treated with 0.8 mM CLBL for 24 hours. At 48 hours post transfection, cells were analyzed for abnormal centriole numbers using DsRed as a transfection marker.

Abnormal centriole duplication driven by each of E2F-1 and HPV-16 E7 was found to be significantly decreased by, 1.7- and 2-fold, respectively (p≦0.05, and p≦0.01, respectively). Surprisingly, treatment with CLBL did not significantly reduce the proportion of cells with abnormal centriole numbers in cells expressing c-Myc (FIG. 3c). This indicates that c-Myc induced abnormal centriole synthesis is significantly less sensitive to proteasome inhibition by CLBL than are other oncogenic stimuli.

Subversion of the cellular ubiquitin/proteasome machinery shown herein revealed surprising differences between these different oncogenic stimuli. Centrosome duplication depends on proteolytic steps involving the ubiquitin/proteasome machinery (Freed, 1999 Genes Dev 13, 2242-57). Several components of the 26S proteasome and E3 ubiquitin ligases localize to the centrosome (Fabunmi, et al. 2000 J Biol Chem 275, 409-13) and have been implicated in initiation of centrosome duplication in late G1. Proteasome inhibitor CLBL was found herein to significantly reduce centriole abnormalities in HPV-16 E7 and E2F-1 transfected cells, however, CLBL was much less efficient in blocking centriole duplication errors in cells expressing c-Myc (FIG. 3c).

To investigate these differences, alternative proteolytic pathways involved in c-Myc induced centrosome duplication errors were investigated. The ubiquitin/proteasome system is functionally impaired in c-Myc expressing Burkitt's lymphoma cells (Gavioli, et al. 2001 Nat Cell Biol. 3, 283-8). c-Myc has been shown to compensate for decreased proteasome-mediated proteolysis, and to activate alternative proteolytic pathways (Gavioli, et al. 2001 Nat Cell Biol. 3, 283-8), including up-regulation of a cytoplasmic oligopeptidase TPPII (Geier, et al. 1999 Science 283, 978-81).

The physiological functions of TPPII have yet to be established, although a membrane-bound isoform has been shown to inactivate the neurohormonee cholecystokinin in the brain (Rose, 1996 Nature 380, 403-9). TPPII has broad tissue distribution, and may play a role in protein degradation by the proteasome system (Wang, et al. 2000 Proc Natl Acad Sci USA 97, 9990-5). Cells with a compromised poteasome system and that overexpress TPPII are able to degrade ubiquitinated proteins, assemble MHC class I molecules and control the cell cycle (Glas, et al. 1998 Nature 392, 618-22). These reports indicate that alternative proteolytic pathways are important for cell viability upon impairment of the proteasome in cells overexpressing c-Myc (Gavioli, et al. 2001 Nat Cell Biol. 3, 283-8). However, it has not been known whether TPPII contributes to malignant transformation.

### Example 5

### c-Myc Induced Abnormal Centriole Synthesis Requires Tripeptidyl Peptidase II (TPPII) Activity

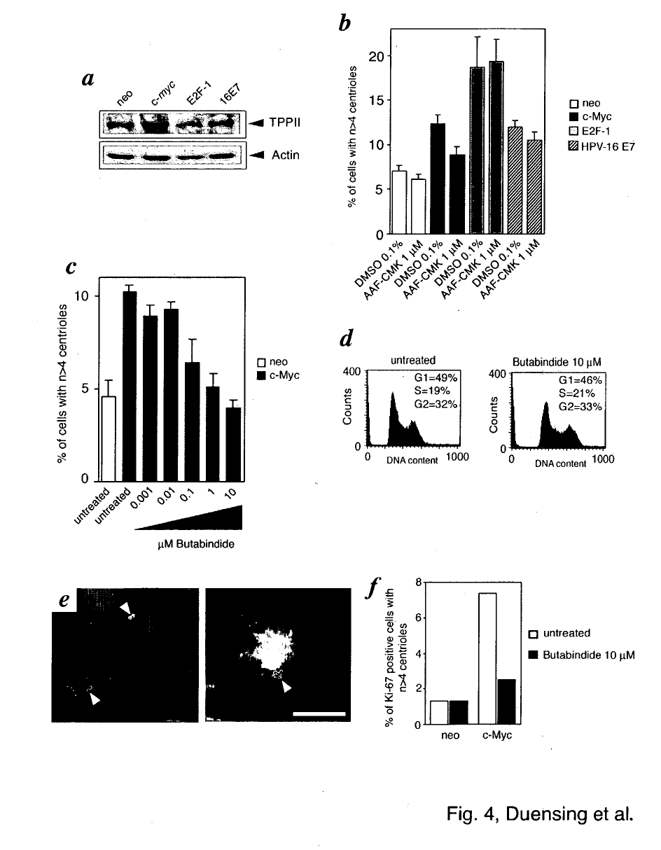

Since Examples herein indicate that c-Myc induced abnormal centriole synthesis has a reduced sensitivity to proteasome inhibition, possible involvement of alternative proteolytic pathways in this process were investigated (FIG. 4).

Tripeptidyl peptidase II (TPPII) is a large cytoplasmic oligopeptidase that cleaves tripeptides from the free N terminus of short polypeptides (Tomkinson, 1999 Trends Biochem Sci. 24, 355-9). The physiologic function TPPII is unclear, however TPPII has been reported to compensate at least in part for loss of proteasome function in certain cell types (Wang, et al. 2000 Proc Natl Acad Sci USA 97, 9990-5).

It is here found that transient overexpression of c-myc, but not E2F-1 and HPV-16 E7, in U20S cells induced an increase of TPPII protein levels detected by immunoblotting (FIG. 4a). Because of these results, a possible interference with TPPII activity by pharmacological inhibitors as a means to modulate abnormal centriole duplication by c-Myc compared to other oncogenic stimuli was therefore examined.

U20S/centrin-GFP cells were transiently transfected with c-Myc, E2F-1, or HPV-16 E7, and were then treated with the covalent TPPII inhibitor Ala-Ala-Phe-chloromethylketone (AAF-CMK) for 24 hours. Centriole numbers were assessed after a total of 48 hours post transfection in cells selected for expression using DsRed as a transfection marker. Reduction of the proportion of c-Myc expressing cells with abnormal centriole numbers in the presence of 1 and 10 μM AAF-CMK was found (FIG. 4b). The decrease induced by 1 μM AAF-CMK in the c-Myc transfected cell population was found to be statistically significant (p≦0.05), whereas changes observed in either E2F-1 or HPV-16 E7 expressing cells were not significant.

A pharmacological inhibitor selective for TPPII, butabindide (Rose, et al. 1996 Cell 32, 311-5), was also used to block TPPII activity. After transient transfection of c-Myc, treatment of cells with 10 μM butabindide for 24 hours was found to produce a dose-dependent and statistically highly significant reduction (p≦0.001) of the number of c-Myc expressing cells with abnormal centriole numbers (FIG. 4c).

Since centrosome duplication is linked to the cell division cycle, the relationship of this observed effect of butabindide on c-Myc-induced centriole duplication errors to the cell cycle was investigated. It was observed that neither treatment of cells with 10 μM AAF-CMK, nor with 10 μM butabindide, was found to substantially alter the cell cycle profile of U20S/centrin-GFP cells (FIG. 4d).

To further analyze the effect of butabindide on abnormal centriole duplication in possible correlation with the proliferation status of c-Myc expressing cells, an assay system to simultaneously determine cell proliferation and centriole numbers was developed. U20S/centrin-GFP cells were transiently transfected with c-Myc using cyan fluorescent protein (CFP) as a transfection marker. After treatment with 10 μM butabindide for 24 hours, cells were stained for the proliferation marker Ki-67 (Gerdes, et al. 1983 Int J Cancer 13-20), and were analyzed by immunofluorescene microscopy. It was observed that treatment with butabindide caused a decrease in the number of c-Myc expressing cells with abnormal centriole numbers, which were mostly Ki-67 positive. In contrast, major increases of Ki-67 negative growth arrested cells were not observed.

It is here shown that c-Myc induces abnormal numbers of centriole in a manner that depends on TPPII activity. TPPII protein levels are upregulated in c-Myc expressing cells even after transient overexpression (FIG. 4a). Inhibition of TPPII using the TPP inhibitor AAF-CMK was found to decrease abnormal centriole numbers induced by c-Myc but not by E2F-1 or HPV-16 E7 (FIG. 4b). Moreover, a selective pharmacological TPPII inhibitor, butabindide, completely blocks abnormal centriole duplication induced by c-Myc in a dose-dependent manner. In contrast to proteasome inhibitors (Kisselev, et al. 2001 Chem Biol 8, 739-58), butabindide suppresses abnormal centriole duplication without imposing cell cycle arrest. These results indicate that there are rate-limiting proteolytic steps during centrosome duplication for which TPPII activity is essential in c-Myc expressing cells.

In conclusion, c-Myc induced centriole duplication errors were significantly and selectively reduced by abrogation of TPPII activity using the pharmacological inhibitor butabindide, without imposing a major cell cycle arrest.

### Example 6

### Maintenance of Centrosome Duplication in Burkitt's Lymphoma Cells Adapted to Proteasome Inhibition

Impaired function of the proteasome usually has a lethal effect on cells (Ciechanover, 2000 Bioessays 22, 442-51). However, adaptation to loss of proteasome activity has been described in certain cell types (Glas, et al. 1998 Nature 392, 618-22). These cells initially die in the presence of proteasome inhibitors but subpopulations of cells resume to grow at otherwise lethal concentrations. It has been demonstrated that in these adapted cells, overexpression of TPPII is critically involved in cell viability and maintenance of ubiquitin-dependent proteolysis (Wang, et al. 2000 Proc Natl Acad Sci USA 97, 9990-5).

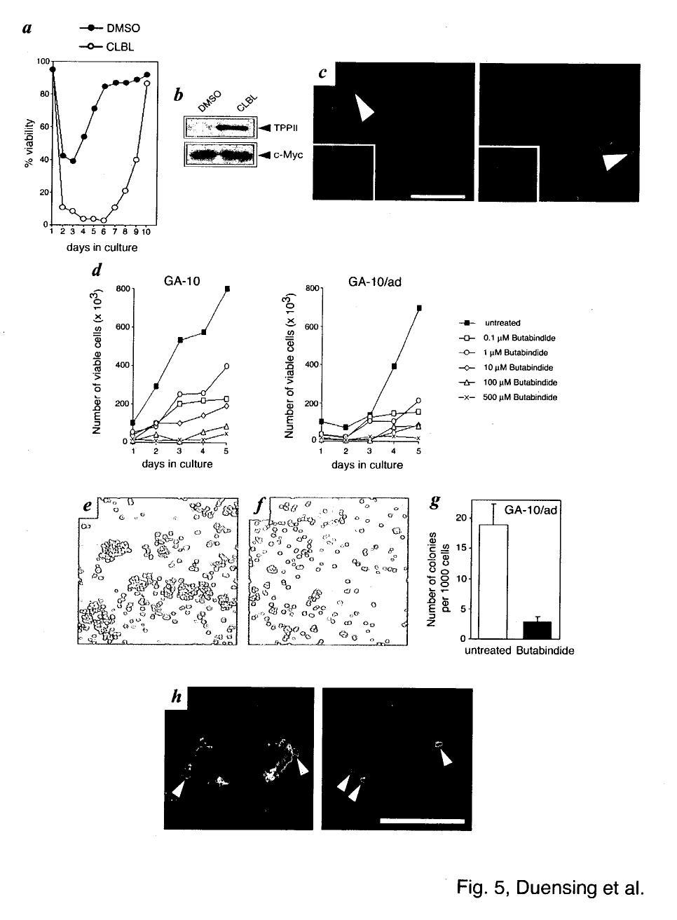

Based on observations herein that TPPII is required for c-Myc induced abnormal centriole duplication, the effect of growth of cells in the presence of a proteasome inhibitor on altered centrosome numbers, control of centrosome duplication, and cell viability was examined. Cells of a human t(8;14) positive, Epstein-Barr virus (EBV) negative Burkitt's lymphoma cell line GA-10 were used. Cells were grown in the presence of 1 μM CLBL (proteosome inhibitor clastolactacystin β-lactone) or with 0.1% DMSO as a solvent control. A marked decrease of cell viability of cells grown in CLBL was observed, and cell survival was restored within approximately 10 days (FIG. 5a). Higher concentrations of CLBL resulted in massive cell death, and no adapted cell populations were obtained in several experiments. GA-10 cells adapted to CLBL (GA-10/ad cells) exhibited increased protein levels of TPPII (FIG. 5b), while the cellular content of c-Myc remained unchanged.

The proportion of cells with abnormal centrosome numbers was determined using immunofluorescence microscopy for γ-tubulin. It was found that 18.7% of untreated GA-10 cells showed abnormal centrosome numbers. Similar levels of centrosome abnormalities were found in CLBL-adapted cells (19.4%) and control cells growing in the presence of DMSO (19.9%), respectively. This indicates that impaired proteasome function in adapted Burkitt's lymphoma cells does not lead to an inhibition of centrosome duplication errors or to decreased centrosome numbers.

The increased protein levels of TPPII in these cells and the results herein indicate that. TPPII is involved in compensatory mechanisms to sustain centrosome duplication. To test whether this function of TPPII requires centrosomal localization of TPPII, double immunofluorescence for TPPII and γ-tubulin was performed (FIGS. 5c,d). It was found that the intensity of TPPII immunofluorescence was higher in CLBL-adapted GA-10 cells, correlating with the increased protein levels detected by immunoblot (FIG. 5b). No co-localization of TPPII with γ-tubulin was observed.

Centrosome duplication is necessary for orderly progression through mitosis and re-entry in the cell cycle (Hinchcliffe, et al. 2001 Genes Dev 15, 1167-81) in most mammalian cells. Whether TPPII activity is necessary for the growth of CLBL-adapted Burkitt's lymphoma cells was investigated. It was observed that the TPPII inhibitor butabindide reduced the growth of CLBL-adapted GA-10 cells in a dose dependent manner (FIG. 5d). Butabindide significantly suppressed colony formation of CLBL-adapted Burkitt's lymphoma cells in soft agar, demonstrating the critical role of TPPII for the malignant potential of adapted Burkitt's lymphoma cells (FIG. 5e, f, and g). It was further found that butabindide abrogated centrosomal duplication and centrosomal function, as indicated by abrogation of spindle formation in treated cells (FIG. 5h).

Butabindide treatment caused a shift from 44% to 66% in the number of cells observed that had become unable to nucleate a microtubule spindle. If no spindle was formed, then cell division would not proceed in butabindide treated cells, and no proliferation would occur. Thus butabindide not only suppressed centrosome duplication, but also centrosome function, and in this manner butabindide suppresses mitotic chromosome instability in c-Myc expressing cancer cells.

The oligopeptidase TPPII is shown herein to play a crucial role, not only for cell survival and proliferation in proteasome-inhibitor adapted Burkitt's lymphoma cells, but also for the maintenance of centrosome duplication. Inhibition of TPPII activity using the selective inhibitor butabindide suppressed abnormal centrosome duplication, and also abrogated cell growth and colony formation, indicating the significant role of TPPII for the malignant potential of Burkitt's lymphoma cells, and as a target for inhibiting malignant progression.

Populations of the human t(8;14) positive Burkitt's lymphoma cell line GA-10 were adapted to grow in the presence of the proteasome inhibitor CLBL. These adapted cells have increased TPPII protein levels, and show similar levels of numerical centrosome abnormalities compared to controls, suggesting that GA-10 cells with impaired proteasome system are still able to control centrosome duplication. While c-Myc requires TPPII activity to trigger abnormal synthesis under transient conditions, TPPII was found not to localize to the centrosome in adapted cells (FIGS. 5e,f). Therefore, TPPII may support centrosome duplication by indirect mechanisms, rather than by direct proteolytic cleavage of centrosomal proteins. Without being limited by any particular mechanism, it is possible that increased TPPII activity supports residual proteasome function, without participating directly in the highly specific processes of the ubiquitin/proteasome system (Wang, et al. 2000 Proc Natl Acad Sci USA 97, 9990-5).

It is here shown that c-Myc rapidly induces centrosome duplication errors, a process which critically depends on the activation of TPPII. The propensity of c-Myc expressing cells for apoptosis may explain the selection pressure to inactivate p53, for example by chromosomal loss (FIG. 1a), or to attenuate the pro-apoptotic activity of c-Myc by mutations in certain areas of the c-myc gene locus (Kuttler, 2001 Oncogene 20, 6084-94). It is shown herein that genomic destabilization can be linked to alternative proteolytic pathways, downstream of the ubiquitin/proteasome machinery.

The role of TPPII for c-Myc induced centrosome duplication errors makes it an attractive target to suppress mitotic infidelity in c-Myc overexpressing tumors. Butabindide, an agent which was originally designed to inhibit TPPII-mediated inactivation of the neurohormone cholecystokinin (Rose, et al. 1996 Nature 380, 403-9) for treatment of obesity, is shown here to have antineoplastic activities. Butabindide can significantly reduce growth and soft agar colony formation of c-Myc expressing Burkitt's lymphoma cells adapted to a compromised proteasome function (FIGS. 5e-g). TPPII inhibitors can have therapeutic or preventive potential to target chromosome number instability, malignant progression, and chemotherapy resistance of c-Myc expressing malignancies.

### Example 7

### Testing of Inhibition of c-Myc Promoted Phenotype by Additional Butabindide Compounds

Each of a set of butabindide compounds, the structure and synthesis of which are shown in U.S. Pat. No. 6,335,360 and are generically described by formula (I) herein, are tested for ability to inhibit c-Myc promoted tumor-associated functions of cells in culture, such as abnormal centriole synthesis, and proliferation in soft agar, using the methods and assays shown herein. The compounds to be tested include UCL 1371 (Rose et al., Nature 380, 403-9).

It is contemplated that a number of butabindide compounds such as UCL 1371 have the ability of to inhibit c-Myc functions of cells in culture, and that the extent of this ability will correlate with inhibition of TPPII, as measured by the respective Kᵢ values of these compounds. These data are significant, as some of these compounds may have favorable properties in a cancer patient in vivo, the properties being absorption and oral availability, distribution to various organs, metabolism and excretion which affect pharmacological lifetime, and toxicity.

### Example 8

### Testing of Butabindide Compounds in an Animal Model System

Butabindide and other TPPII inhibitors are tested in an animal model for lymphoma. Animal models for lymphoma are known for example, a Burkitt lymphoma mouse model based on cells carrying an engineered BL-specific myc translocation breakpoint (Kovalchu, et al. 2000 J Exper Med 192, 1183-90); an anaplastic large cell lymphoma (ALCL) model established by interleukin (IL)-9 transfection (Bittner, et al. 2000 Lab Inves 80 (10)); and a miniature swine model of Post Transplant Lymphoproliferative Disease (Sachs, D. et al. Massachusetts General Hospital, Boston Mass.).

Accordingly, butabindide and butabindide compounds identified in Example 7 will be used in animal model systems. Groups of mice (at least 6 in each group) are identified to be treated as follows: the experimental groups will be treated with the compound identified herein, the negative control groups will be untreated (administered vehicle only), and the positive control groups will be administered a classical chemotherapeutic agent. At least two different experimental groups of animals will be established to test at least two different doses of the compound identified herein.

Data to be collected include tumor sizes and presence or absence of tumors. Administration of a suitable compound, for example, butabindide, dissolved in an appropriate vehicle will be found to delay the appearance of new tumors and shrink or inhibit further growth of an existing tumor, and prolong the natural lifespan of the affected animal, in comparison to an otherwise identical animal not administered the compound (administered vehicle only). Statistical significance (P values) will be determined from Mantel-Cox tests performed on Kaplan-Meier survivor functions. The positive control group will be administered a known anti-cancer agent previously used in treatment of lymphoma, such as doxorubicin, cyclophosphamide, Velcade, or vincristine.

### Example 9

### Testing a Butabindide Compound in Combination with a Classical Anticancer Agent in an Animal Model System

As anti-tumor agents are frequently used in combination, such as the aforementioned doxorubicin, cyclophosphamide, Velcade, and vincristine which may be used with each other or with another agent such as cis-platin, the butabindide compound identified herein is combined with other anti-tumor agents to develop a protocol most efficacious against lymphoma.

A major problem in current anti-cancer chemotherapeutic regimens is develoment of drug resistance during the course of a prolonged chemotherapeutic regiment. As butabindide and related compounds are based on inhibition of a novel target, TPIII, cells that have developed resistance to a DNA cross-linking agent, a DNA intercalating agent, or a nucleoside analog, may be fully sensitive to butabindide or a butabindide compound, which is unrelated both structurally and on the basis of mechanism of action.

What is claimed is:

1. A method of reducing centrosome duplication errors in a cell, comprising treating the cell with an effective dosage of an inhibitor of a tripeptidyl peptidase II (TPPII).

2. The method of claim 1, wherein the inhibitor is butabindide.

3. The method of claim 1, wherein the inhibitor is a derivative of butabindide.

4. The method of claim 1, wherein the cell is a cancer cell.

5. The method of claim 4, wherein the cancer cell contains a centrosome abnormality.

6. The method of claim 4, wherein the cancer is malignant.

7. The method of claim 4, wherein the cell has altered expression or activity of a c-myc oncogene.

8. The method of claim 4, wherein the cancer is selected from the group consisting of lymphoma, leukemia, lung, colorectal carcinoma, head and neck, skin, prostate, breast, melanoma, ovarian, brain, esophageal, gastric, and liver.

9. A method of inhibiting growth of a lymphoma cell, comprising administering an inhibitor of a tripeptidyl peptidase II to a subject having a lymphoma.

10. The method of claim 9, wherein the cell is selected from the group of lymphomas consisting of B and T cell lymphomas.

11. The method of claim 9, wherein the cell is selected from the group of lymphomas consisting of Hodgkin's, non-Hodgkins, and Burkitt's.

12. The method of claim 9, wherein the cell is Burkitt's lymphoma.

13. A method of decreasing viability of a lymphoma cell, comprising treating the cell with an effective dosage of an inhibitor of a tripeptidyl peptidase II.

14. The method of claim 13, wherein the inhibitor is a butabindide compound.

15. The method of claim 13, wherein the inhibitor is selected from the group consisting of UCL1371 and butabindide.

16. The method of claim 13, wherein the inhibitor is butabindide.

17. The method of claim 13, wherein the dosage is at least about 100 micromolar.

18. A method of identifying an anti-tumor agent, comprising screening for an inhibitor of TPPII.

19. A method of treating a cancer cell, comprising contacting the cell with a TPPII inhibitor.

20. The method of claim 19, wherein the cancer cell carries a myc mutation.

21. The method of claim 19, wherein the cancer cell is a lymphoma cell.

22. The method of claim 19, wherein the TPPII inhibitor is a butabindide compound.

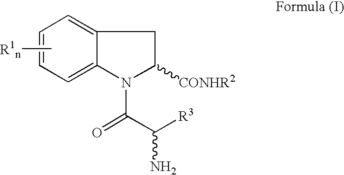

23. The method of claim 22, wherein the butabindide compound comprises a formula (I) compound wherein each of the number of n R¹ groups (covalently attached to the 6membered ring of the indoline moiety) may be the same or different, and is selected from the group consisting of halogen, OH; C₁-C₆ alkyl optionally substituted by one or more radicals selected from the group consisting of halogen and OH; (C₁-C₆) alkenyl optionally substituted by one or more radicals selected from the group consisting of halogen and OH; (C₁-C₆) alkynyl, optionally substituted by one or more radicals selected from the group consisting of halogen and OH, X(C₁-C₆)alkyl, wherein X is S, O, or OCO, and the alkyl is optionally substituted by one or more radicals selected from the group consisting of halogen and OH; SO₂(C₁-C₆)alkyl, optionally substituted by at least one halogen, YSO₃H, YSO₂(C₁-C₆)alkyl, wherein Y is O or NH and the alkyl is optionally substituted by at least one halogen, a diradical —X¹—(C₁-C₆)alkylene-X¹ is O or S; and a benzene ring fused to the indoline ring; n is from 0 to 4; R² (at the carboxyamide end of the molecule) is CH₂R⁴, wherein R⁴ is C₁-C₆ alkyl substituted by one or more radicals selected from the group consisting of halogen and OH; (CH)ₚZ(CH₂)_{q}CH₃, wherein Z is O or S, p is from 0 to 5 and q is from 0 to 5; (C₁-C₆) unsaturated alkyl; or (C₃-C₆) cycloalkyl; or R2 is (C₁-C₆)alkyl or O(C₁-C₆)alkyl, each optionally substituted by at least one halogen; R³ at the amino end of the molecule is H; (C₁-C₆)alkyl optionally substituted by at least one halogen; (CH2)ₚZR⁵ wherein p is from 1 to 3, Z is O or S and R⁵ is H or (C₁-C₆)alkyl; benzyl; or a pharmaceutically acceptable acid addition salt thereof; provided that when R³ is a halogen atom, a O—(C₁-C₆)alkyl; OH or (C₁-C₄)alky group; R² is CH₂R⁴ wherein R⁴ is (CH₂)₂SCH₃, —(CH₂)₂OH or cyclohexyl; or R² is a (C₁-C₆)alkyl group; then R³ is neither a hydrogen atom nor a (C₁-C₄)alkyl group.

24. A method of diagnosing a precancerous or cancerous cell, comprising identifying a cell having centrosomal abnormalities wherein growth of the cell is inhibited by a TPPII inhibitor.

25. The method of claim 24, wherein the centrosomal abnormalities comprise the cell having a greater number of centrosomes or mitotic spindle poles compared to a control normal cell.

26. The method of claim 24, wherein the TPPII inhibitor is selected from the group consisting of a butabindide compound, AAF-CMK, AAF-MCA, and UCL1371.

27. The method of claim 24, wherein the TPPII inhibitor is butabindide.

28. A method of prognosis of susceptibility of test cell to treatment with a TPPII inhibitor, the method comprising
determining frequency in the cell of centrosomal abnormalities compared to that of a normal control cell, wherein the test cell is in need of diagnosis and prognosis for a cancer or a precancerous condition; and
determining growth rate of the cell in the presence of the TPPII inhibitor compared to growth rate of the control cell, wherein a greater frequency of centrosomal abnormalities and an inhibition of the growth rate by the TPPII inhibitor in the test cell compared to the normal cell indicates that the test cell is cancerous or precancerous, and that the cancer can be treated with a TPPII inhibitor.

29. A method of inhibiting progression of cancer in a precancerous cell, comprising contacting the cell with a TPPII inhibitor.

30. The method of claim 29, wherein the TPPII inhibitor is a butabindide compound.

31. The method of claim 29, wherein the TPPII inhibitor is butabindide.

32. A composition for use in treating a cancerous or precancerous condition comprising a TPPII inhibitor and an anti-cancer agent.

33. The composition of claim 32, wherein the anti-cancer agent is a cis-platin, a taxol, an irinotecan, a Velcade, or a Gleevec.
